(19) United States
(12) Patent Application Publication
(10) Pub. No.: US 2011/0056508 A1
(43) Pub. Date: Mar. 10, 2011

(54) BLEACHING AGENT COMPRISING CATIONIC 3,4-DIHYDROISOQUINOLINE DERIVATIVES, SPECIAL ALKANOL AMINES AND HYDROGEN PEROXIDE

(76) Inventors: Wibke Gross, Hueckelhoven (DE); Ralph Nemitz, Juechen (DE); Denise Fuhr, Duesseldorf (DE); Dorota Sendor-Müller, Duesseldorf (DE); Kristin Miehlich, Wuppertal (DE); Georg Knuebel, Duesseldorf (DE)

(21) Appl. No.: 12/943,422

(22) Filed: Nov. 10, 2010

Related U.S. Application Data
(63) Continuation of application No. PCT/EP2009/051834, filed on Feb. 17, 2009.

(30) Foreign Application Priority Data
May 16, 2008 (DE) 10 2008 024 030.3

(51) Int. Cl. A61K 8/18; A61K 8/49; A61Q 5/08

(52) U.S. Cl. 132/208; 424/62

(57) ABSTRACT

Agent for bleaching keratinic fibers containing hydrogen peroxide, at least one cationic 3,4-dihydroisoquinoline derivate of general structure (I)—

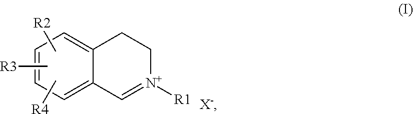

and at least one alkanol amine of the following general structure (II)—

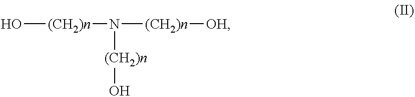

where n is a natural number from 2 to 6. The invention further relates to use of the agent for bleaching hair and to a corresponding method.

### CROSS REFERENCE TO RELATED APPLICATIONS

The present application is a continuation of International Patent Application No. PCT/EP2009/051834 filed 17 Feb. 2009, which claims priority to German Patent Application No. 10 2008 024 030.3 filed 16 May 2008, both of which are incorporated herein by reference.

The present invention relates to agents for bleaching keratin fibers, that is, agents for use on keratin fibers, particularly human hair, containing cationic 3,4-dihydroisoquinoline derivatives, special alkanol amines and hydrogen peroxide, to the use of this combination for bleaching hair, and to a corresponding method.

Changing the shape and color of the hair is an important area of modern cosmetics. It allows the appearance of hair to be adapted to the latest fashion trends and personal preferences of the individual. Permanent wave methods and other methods of changing the shape of hair can be used almost regardless of the type of hair being treated. In contrast, dyeing and bleaching methods are limited to certain natural hair colors. The basic principles of bleaching methods are known to one skilled in the art and can be researched in relevant monographs such as Kh. Schrader, Grundlagen and Rezepturen der Kosmetika, 2^{nd} Ed., Dr. Alfred Hüthig Verlag, Hei-delberg (1989), or W. Umbach (Ed.), Kosmetik, 2^{nd} Ed., Georg Thieme Verlag, Stuttgart, New York (1995).

Conventional hair coloring agents generally include at least one developer substance and at least one coupler substance, and optionally also contain direct dyes as tints. Coupler and developer components are also known as oxidation dye intermediates.

In addition to dyeing their hair, many consumers choose to bleach or lighten their natural hair color because blonde hair is considered to be attractive and desirable from a fashion perspective. Various bleaching agents with varying bleaching power are commercially available for this purpose. Oxidizing agents found in these products have the ability to lighten hair fiber by oxidative breakdown of the hair's natural pigment melanin. For a moderate bleaching effect, the use of hydrogen peroxide—optionally with the use of ammonia or other alkalizing agents—is sufficient by itself as an oxidizing agent. For a stronger bleaching effect, a mixture of hydrogen peroxide and peroxodisulfate salts and/or peroxomonosulfate salts is conventionally used. Bleaching is, however, also associated with damage to the hair, as not only is the natural coloring components of the hair damaged by oxidation, but also other structural components of the hair are damaged. The extent of damage can range from coarse, brittle and tangled hair, through reduced resistance and breaking strength of the hair, to breakage of the hair. The larger the amount of hydrogen peroxide and optional peroxodisulfates used, the greater the damage caused to the keratin fibers. Hair coloring or bleaching agents that provide good bleaching power without damaging hair fibers at the same time have hitherto been unknown.

Before being used on human hair, hair coloring and/or bleaching agents in solid or paste form are mixed with dilute aqueous hydrogen peroxide solution. This mixture is applied to the hair and then rinsed out after a certain contact time. Contact time on the hair required to achieve complete pigment removal or bleaching is typically around 30 to 40 minutes. Obviously, there is a need among users of these hair colors or bleaching agents to reduce this contact time.

Bleaching processes on keratin fibers conventionally take place in alkaline pH ranges, particularly from 9.0 to 10.5. These alkaline pH values are necessary to ensure that the external cuticle opens, allowing active species (dye intermediates and/or hydrogen peroxide) to penetrate into the hair. Ammonia is conventionally used as the alkalizing agent, which, however, has a disadvantageous intense odor and possible irritation for the user.

Although bleaching agents hitherto available on the market generally have good bleaching power, they still can be improved with respect to hair damage, length of application times and possible skin irritation caused by high concentrations of oxidizing and alkalizing agents.

Use of cationic isoquinoline derivatives as bleach activators for optimizing the bleaching power of oxidative bleach active ingredients on hair is described in German Patent Application No. 10 2007 047 688.6.

The present invention provides novel agents containing bleach activators for bleaching or lightening hair which, in terms of their bleaching power, are comparable with or superior to that known in the art, while at the same time results in hair damage which is comparable to or ideally less than that in the prior art.

It is known that cationic isoquinoline derivatives in combination with imidazole optimize the bleaching action on the hair. However, use of imidazole in cosmetic agents brings about various disadvantages, especially from a toxicological viewpoint. Accordingly, the present invention further provides a replacement for imidazole as bleach activator which is at least comparable with the prior art.

Unforeseeably, it has now been found that use of a combination of cationic 3,4-dihydroisoquinoline compounds of general structure (I) (provided below), certain alkanol amities of general structure (II) (provided below) and hydrogen peroxide bleaches hair much more strongly than would be possible using cationic 3,4-dihydroisoquinoline compounds and hydrogen peroxide alone or by combination thereof with imidazole.

It was therefore surprisingly discovered that improvement in bleaching effect brought about through use of the combination according to the invention presents an outstanding opportunity to replace the imidazole.

The amount of oxidizing agent used can be reduced because of the improved bleaching power obtained by use of the agent according to the invention, thereby minimizing hair damage. The contact time required to achieve a bleaching effect corresponding to the prior art can also be shortened in this way.

The present invention thus firstly provides an agent for bleaching keratin fibers, wherein it contains in a cosmetic carrier—

at least one cationic 3,4-dihydroisoquinoline derivative of following general structure (I)—

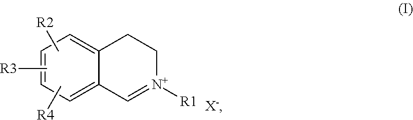

wherein
R1 is a C₁-C₆ alkyl group, C₂-C₆ alkenyl group, C₂-C₆ hydroxyalkyl group, C₁-C₆ alkoxy C₂-C₆ alkyl group, carboxy C₁-C₆ alkyl group, aryl C₁-C₆ alkyl group, C₁-C₆ dialkylamino C₂-C₆ alkyl group, heteroaryl C₁-C₆ alkyl group, 3-oxobutyl group, 2-oxopropyl group, aryl group or heteroaryl group;
R2, R3 and R4 are each independently hydrogen, a hydroxyl group, amino group, di(C₁-C₆)alkylamino group, C₁-C₆ alkoxy group, halogen, nitro group, carboxy group, nitrile group, optionally substituted aryl group, C₂-C₆ alkenyl group, optionally substituted heteroaryl group, or R2 and R3 together can form a further fused carbocyclic or heterocyclic ring which can be saturated or unsaturated and can optionally be substituted by up to three substituents; and
X⁻ is a physiologically tolerable anion;
at least one alkanol amine of the following general structure (II)—

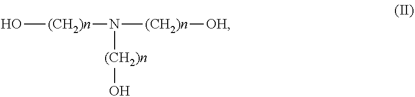

wherein n is a natural number from 2 to 6; and
hydrogen peroxide.

The term “keratin fibers” here refers to fur, wool, feathers and, in particular, human hair. Although agents according to the invention are primarily suitable for dyeing and/or bleaching keratin fibers, there is nothing in principle precluding their use in other fields.

Examples of suitable residues as substituents of compounds of formula (I) are listed below—
Examples of (C₁ to C₆) alkyl residues include the —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, and —C(CH₃)₃ groups.
Examples of (C₁ to C₆) alkoxy residues include —OCH₃, —OCH₂CH₃, —OCH₂CH₂CH₃, —OCH(CH₃)₂, —OCH₂CH₂CH₂CH₃, —OCH₂CH(CH₃)₂, —OCH(CH₃)CH₂CH₃, and —OC(CH₃)₃, particularly a methoxy or an ethoxy group.
Preferred examples of (C₂ to C₆) hydroxyalkyl groups include —CH₂CH₂OH, —CH₂CH₂CH₂OH, —CHCH(OH)CH₃, and —CH₂CH₂CH₂CH₂OH, with the —CH₂CH₂OH group being preferred.
Examples of halogen atoms include F, Cl or Br atoms, with Cl atoms being most particularly preferred.
Examples of a (C₁ to C₄) dialkylamino group include —N(CH₃)₂, —N(CH₂CH₃)₂.
Examples of (C₁ to C₄) alkoxy (C₁ to C₄) alkyl groups include—CH₂CH₂OCH₃, —CH₂CH₂CH₂OCH₃, —CH₂CH₂OCH₂CH₃, —CH₂CH₂CH₂OCH₂CH₃, —CH₂CH₂OCH(CH₃)₂, and —CH₂CH₂CH₂OCH(CH₃)₂.
Examples of a C₂-C₆ alkenyl group include a 2-propenyl group (allyl group), a but-3-enyl group, a but-2-enyl group, a pent-4-enyl group or a pent-3-enyl group. The 2-propenyl group is particularly preferred in this context.
Examples of a carboxy C₂-C₆ alkyl group include the carboxymethyl group, 2-carboxyethyl group or 3-carboxypropyl group.
Examples of a heteroaryl C₁-C₆ alkyl group include the pyridin-2-yl methyl group, pyridin-3-yl methyl group, pyridin-4-yl methyl group, pyrimidin-2-yl methyl group, 1H-pyrrol-1-yl methyl group, 1H-pyrrol-1-yl ethyl group, 1H-pyrazol-1-yl methyl group or 1H-pyrazol-1-yl ethyl group.
An example of aryl groups is the phenyl group.
Examples of aryl (C₁ to C₄) alkyl groups include the benzyl and 2-phenylethyl groups.

Agents according to the invention contain at least three substantial constituents—(1) at least one cationic 3,4-dihydroisoquinoline derivative of the formula (I), (2) at least one alkanol amine of the formula (II), and (3) hydrogen peroxide. Agents according to the invention can also be “application mixtures”, in other words, agents which are packaged separately (for stability reasons, for example) but which are mixed together before use to form an application mixture and then applied.

Preferably, the residue R1 of general structure (I) is a C₁-C₆ alkyl group, C₂-C₆ alkenyl group or C₂-C₆ hydroxyalkyl group.

It is furthermore preferred according to the invention that residues R2, R3 and R4 of general structure (I) are each a hydrogen atom.

It is preferable for X⁻ according to formula (I) to be chosen from halide (chloride, bromide, iodide), benzene sulfonate, p-toluene sulfonate, C₁-C₄ alkane sulfonate, trifluoromethane sulfonate, acetate, trifluoroacetate, perchlorate, ½ sulfate, hydrogen sulfate, tetrafluoroborate, hexafluorophosphate, hexafluorozincate or tetrafluorozincate.

It is particularly preferred according to the invention that the physiologically tolerable anion X⁻ is a halide ion (particularly chloride or bromide), hydrogen sulfate, ½ sulfate, p-toluenesulfonate, benzenesulfonate or acetate.

Particularly preferred cationic 3,4-dihydroisoquinoline derivatives of the general formula (I) include—

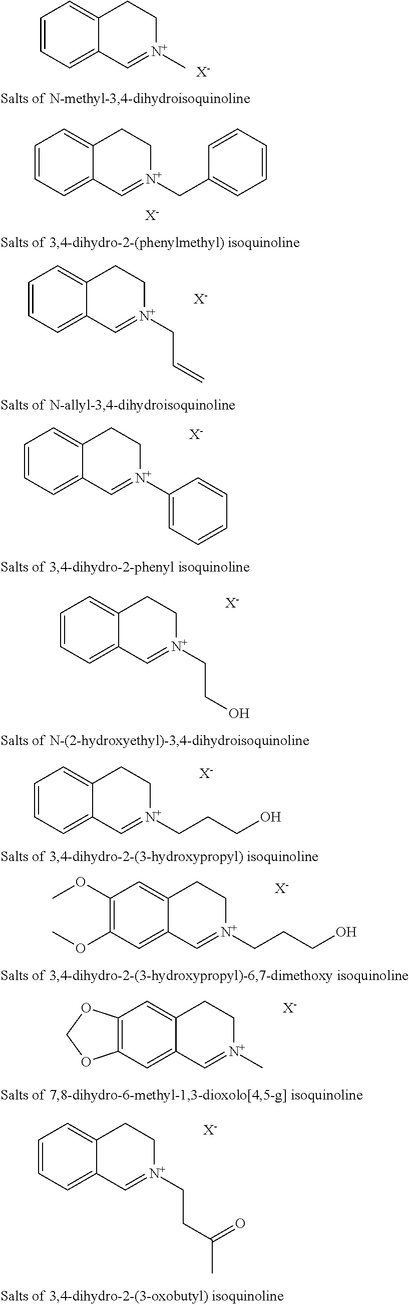

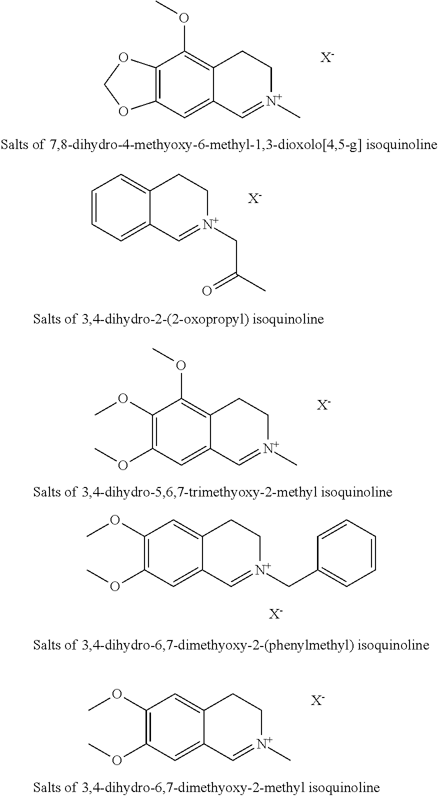

Agents according to the invention are most particularly preferred wherein the cationic 3,4-dihydroisoquinoline derivative of general structure (I) contain at least one compound chosen from N-methyl-3,4-dihydroisoquinoline p-toluenesulfonate, N-methyl-3,4-dihydroisoquinoline benzenesulfonate, N-methyl-3,4-dihydroisoquinoline hydrogen sulfate, N-allyl-3,4-dihydroisoquinoline p-toluenesulfonate, N-allyl-3,4-dihydroisoquinoline benzenesulfonate, N-allyl-3,4-dihydroisoquinoline bromide, N-allyl-3,4-dihydroisoquinoline acetate, 3,4-dihydro-2-(3-hydroxypropyl)isoquinoline p-toluenesulfonate, 3,4-dihydro-2-(3-hydroxypropyl)isoquinoline benzenesulfonate, 3,4-dihydro-2-(3-hydroxypropyl)isoquinoline bromide, 3,4-dihydro-2-(3-hydroxypropyl)isoquinoline acetate, 3,4-dihydro-2-(2-hydroxyethyl)isoquinoline p-toluenesulfonate, 3,4-dihydro-2-(2-hydroxyethyl)isoquinoline benzenesulfonate, 3,4-dihydro-2-(2-hydroxyethyl)isoquinoline bromide or 3,4-dihydro-2-(2-hydroxyethyl)isoquinoline acetate.

Various trialkanol amines having a chain length from 2 to 6 C atoms can be used as alkanol amines. Preferably, n is a whole number from 2 to 4.

It is particularly preferable for the alkanol amine of general formula (II) to be triethanolamine (alternative name: tris(2-hydroxyethyl)amine).

Unless explicitly stated otherwise, the amounts given below refer to total weight of the ready-to-use agent.

As the first substantial component, agents according to the invention preferably contain cationic 3,4-dihydroisoquinoline derivatives of general structure (I) in an amount from 0.03 to 65.00 mmol, particularly from 1.00 to 40.00 mmol, based on 100 g of the ready-to-use agent.

As the second substantial component, agents according to the invention preferably contain alkanol amines of general formula (II) in an amount from 0.03 to 65.00 mmol, particularly from 1.00 to 40.00 mmol, based on 100 g of the ready-to-use agent.

As the third substantial component, hydrogen peroxide is present in the agent. Hydrogen peroxide is preferably used as an aqueous solution; however, it can also be used in the form of a solid addition compound of hydrogen peroxide with inorganic or organic compounds such as sodium perborate, sodium percarbonate, magnesium percarbonate, sodium percarbamide, polyvinyl pyrrolidone n H₂O₂ (n is a positive whole number greater than 0), urea peroxide and melamine peroxide.

Most particularly preferred according to the invention are aqueous hydrogen peroxide solutions. Concentration of hydrogen peroxide solutions is determined by legal requirements as well as by the desired effect, with 6 to 12 percent solutions in water preferably used. Preferred agents contain—relative to their weight—0.5 to 12 wt. %, preferably 2 to 10 wt. %, particularly preferably 3 to 6 wt. %, of hydrogen peroxide (calculated as 100% H₂O₂).

Bleaching processes on keratin fibers conventionally take place in an alkaline environment. In order to protect the keratin fibers as well as the skin as much as possible, it is preferred not to have too high a pH. Therefore, the pH of the ready-to-use agent is preferably from 7 to 11, particularly 8 to 10.5. Within the respect to the present invention, pH values refer to pH values measured at a temperature of 22° C.

If the amount of at least one alkanol amine of general structure (II) has not yet established a pH within the desired range, further alkalizing agents from the group formed by ammonia, basic amino acids, alkali hydroxides, alkali metal metasilicates, alkali phosphates and alkali-hydrogen phosphates can be used to establish the desired pH. Preferred alkali metal ions include lithium, sodium, and potassium, particularly sodium or potassium. Basic amino acids which can be used as alkalizing agents according to the invention are preferably chosen from L-arginine, D-arginine, D,L-arginine, L-lysine, D-lysine, and D,L-lysine, with L-arginine, D-arginine, D,L-arginine being particularly preferable as an alkalizing agent. Alkali hydroxides which can be used as the alkalizing agent according to the invention are preferably chosen from sodium hydroxide and potassium hydroxide.

For strong bleaching of very dark hair, use of hydrogen peroxide or its addition products with organic or inorganic compounds is often not sufficient. Here, a combination of hydrogen peroxide and persulfates is often used. It has been found that mixing the 3,4-dihydroisoquinoline compounds of general formula (I) together with alkanol amines of formula (II) results in an increase in bleaching capacity, not only with hydrogen peroxide alone but also with a combination of hydrogen peroxide and persulfate salts.

Should the consumer desire a very strong bleaching effect, in a further embodiment at least one inorganic persulfate salt can be additionally included in the agent for bleaching keratin fibers, in addition to the cationic 3,4-dihydroisoquinoline compound of general structure (I), alkanol amines of formula (II) and hydrogen peroxide.

Persulfate salts can be included in an amount from 0.1 to 25 g, particularly from 1 to 15 g, based on 100 g of the ready-to-use agent.

Preferred persulfate salts include ammonium peroxodisulfate, potassium peroxodisulfate and sodium peroxodisulfate (alternative names: ammonium persulfate, potassium persulfate and sodium persulfate).

As previously mentioned, agents according to the invention can also be prepared directly before application from two or more separately packaged preparations. This allows separation of incompatible ingredients, preventing a premature reaction.

A conventional route thus involves mixing a first agent containing the cationic 3,4-dihydroisoquinoline compounds of general formula (I) and the alkanol amine of general formula (II) together with a second agent containing oxidizing agent(s) according to the invention just before use.

The present invention therefore also provides an agent for bleaching keratin fibers, particularly human hair, which is obtained just prior to application to the hair from a free-flowing preparation A containing the cationic 3,4-dihydroisoquinoline compounds of general formula (I) as well as an alkanol amine of general formula (II) and an oxidizing agent preparation B containing at least one oxidizing agent chosen from hydrogen peroxide and/or its addition compounds with organic or inorganic compounds.

The oxidizing agent preparation B is preferably an aqueous, free-flowing oxidizing agent preparation. Preferred agents according to the invention for bleaching keratin fibers includes those wherein the free-flowing oxidizing agent preparation B—relative to its weight—contains 40 to 90 wt. %, preferably 50 to 85 wt. %, particularly preferably 55 to 80 wt. %, more preferably 60 to 77.5 wt. %, and in particular 65 to 75 wt. % of water.

Use of persulfate salts generally takes place in the form of an optionally dedusted powder or a pressed molding. To prevent premature degradation of the 3,4-dihydroisoquinoline derivatives from contact with the persulfates, preferably the persulfates are provided as a separately packaged component C.

In this connection, the present invention also provides an agent for bleaching human hair consisting of three components. This agent is prepared just prior to application to the hair by careful mixing of a free-flowing preparation A containing the cationic 3,4-dihydroisoquinoline compounds of general formula (I) and an alkanol amine of general formula (II), an oxidizing agent preparation B containing at least one oxidizing agent chosen from hydrogen peroxide and/or its addition compounds with organic or inorganic compounds, and additionally a third preparation C in powder form containing at least one inorganic persulfate salt.

Mixing preparations A and B or optionally preparations A, B and C before application provides an application mixture which is an agent according to the invention containing the three necessary constituents.

An emulsifier or surfactant can preferably be added to the free-flowing preparations A and/or B; surface-active substances being referred to as surfactants or as emulsifiers depending on area of application and chosen from anionic, cationic, zwitterionic, ampholytic and non-ionic surfactants and emulsifiers. These substances are described below in detail.

It has also proved advantageous for coloring and/or bleaching agents according to the invention to contain non-ionogenic interfacially-active substances. Interfacially-active substances having an HLB value of 5.0 or more are preferred.

Due to their easy processability, particularly preferred non-ionogenic surface-active substances are commercially obtainable in pure form as solids or liquids. In this context, purity does not refer to chemically pure compounds. Instead, mixtures of various homologs can be used, particularly if they are naturally-based products, for example, with various alkyl chain lengths such as those obtained in products based on natural fats and oils. Alkoxylated products, too, are usually mixtures with varying degrees of alkoxylation. Purity in this context refers rather to the fact that the chosen substances should preferably be free from solvents, adjusters and other accompanying substances.

Preferred non-ionogenic interfacially-active substances include—
alkoxylated fatty alcohols having 8 to 22, in particular 10 to 16, carbon atoms in the fatty alkyl group and 1 to 30, in particular 1 to 15, ethylene oxide and/or propylene oxide units. Preferred fatty alkyl groups include lauryl, myristyl, cetyl groups but also stearyl, isostearyl and oleyl groups; preferred compounds of this class are lauryl alcohol having 2 to 4 ethylene oxide units, oleyl and cetyl alcohol each having 5 to 10 ethylene oxide units, cetyl and stearyl alcohol and mixtures thereof having 10 to 30 ethylene oxide units, and the commercial product Aethoxal® B (Henkel), a lauryl alcohol having 5 ethylene oxide and 5 propylene oxide units. In addition to conventional alkoxylated fatty alcohols, end-capped compounds can also be used according to the invention. In these compounds the alkoxy group does not have an OH group at the end but instead is “capped” in the form of an ether, particularly a C₁ to C₄ alkyl ether. One example of such a compound is the commercial product Dehypon® LT 054, a C₁₂₋₁₈ fatty alcohol+4.5 ethylene oxide butyl ether;
alkoxylated fatty acids having 8 to 22, in particular 10 to 16, carbon atoms in the fatty acid group and 1 to 30, in particular 1 to 15, ethylene oxide and/or propylene oxide units. Preferred fatty acids include lauric, myristic, palmitic, stearic, isostearic and oleic acid;
alkoxylated, preferably propoxylated, and particularly ethoxylated, mono-, di- and triglycerides. Examples of preferred compounds are glycerol monolaurate+20 ethylene oxide and glycerol monostearate+20 ethylene oxide;
polyglycerol esters and alkoxylated polyglycerol esters. Preferred compounds of this class include poly(3)glycerol diisostearate (commercial product: Lameform® TGI (Henkel)) and poly(2)glycerol polyhydroxystearate (commercial product: Dehymuls® PGPH (Henkel));
sorbitan fatty acid esters and alkoxylated sorbitan fatty acid esters such as sorbitan monolaurate and sorbitan monolaurate+20 ethylene oxide (EO); and
alkyl phenols and alkyl phenol alkoxylates having 6 to 21, in particular 6 to 15, carbon atoms in the alkyl chain and 0 to 30 ethylene oxide and/or propylene oxide units (for example nonyl phenol+4 EO, nonyl phenol+9 EO, octyl phenol+3 EO and octyl phenol+8 EO).

Particularly preferred classes of non-ionogenic interfacially-active substances include alkoxylated fatty alcohols, alkoxylated fatty acids, as well as alkyl phenols and alkyl phenol alkoxylates.

Agents according to the invention containing non-ionogenic interfacially-active substances in amounts from 1 to 5 wt. % have proved to be particularly advantageous.

Bleaching agents according to the invention can further contain all active ingredients, additives and auxiliary substances known in such preparations. Often the agents contain at least one surfactant, with anionic and zwitterionic, ampholytic, non-ionic and cationic surfactants being suitable in principle. It has proved advantageous in many cases, however, to choose surfactants from anionic, cationic or non-ionic surfactants. Anionic surfactants can be most particularly preferred here.

Preferred anionic surfactants include alkyl sulfates, ether carboxylic acid salts having 10 to 18 C atoms in the alkyl group and up to 12 glycol ether groups in the molecule, such as C₁₂H₂₅(C₂H₄O)₆CH₂COONa, and particularly salts of saturated and especially unsaturated C₈ to C₂₂ carboxylic acids such as oleic acid, stearic acid, isostearic acid and palmitic acid.

These anionic surfactants should preferably be in solid form, particularly in powder form. Soaps which are solid at room temperature are most particularly preferred, particularly sodium stearate. These are preferably present in amounts from 5 to 20 wt. %, particularly 10 to 15 wt. %.

In particular, C₈ to C₂₂ alkyl mono- and oligoglycosides and ethoxylated analogs thereof are suitable as non-ionic surfactants. In particular, non-ethoxylated compounds have proven to be particularly suitable.

Examples of cationic surfactants which can be used in hair bleaching agents according to the invention include quaternary ammonium compounds. Ammonium halides are preferred, such as alkyl trimethylammonium chlorides, dialkyl dimethylammonium chlorides and trialkyl methylammonium chlorides, for example, cetyl trimethylammonium chloride, stearyl trimethylammonium chloride, distearyl dimethylammonium chloride, lauryl dimethylammonium chloride, lauryl dimethyl benzyl ammonium chloride and tricetyl methylammonium chloride. Other cationic surfactants which can be used according to the invention are quaternized protein hydrolysates.

Compounds having alkyl groups which are used as surfactants can each be uniform substances. It is generally preferable, however, to use native vegetable or animal raw materials as starting products for these substances so that mixtures of substances having differing alkyl chain lengths (depending on the individual raw material) are obtained.

As a further component, compositions according to the invention can contain at least one ammonium compound from ammonium chloride, ammonium carbonate, ammonium bicarbonate, ammonium sulfate and/or ammonium carbamate in an amount of from 0.5 to 10, preferably 1 to 5 wt. %, based on overall composition of the agent.

Coloring and/or bleaching agents according to the invention can also contain additional active ingredients, auxiliary substances and additives, such as non-ionic polymers (e.g., vinyl pyrrolidone/vinyl acrylate copolymers, polyvinyl pyrrolidone and vinyl pyrrolidone/vinyl acetate copolymers and polysiloxanes), cationic polymers (e.g., quaternized cellulose ethers, polysiloxanes having quaternary groups, dimethyldiallyl ammonium chloride polymers, acrylamide-dimethyldiallyl-ammonium chloride copolymers, dimethylaminoethyl methacrylate-vinyl pyrrolidone copolymers quaternized with diethyl sulfate, vinyl pyrrolidone-imidazoline-methochloride copolymers and quaternized polyvinyl alcohol), zwitterionic and amphoteric polymers (e.g., acrylamidopropyl trimethylammonium chloride/acrylate copolymers and octylacrylamide/methyl methacrylate/tert-butyl aminoethyl methacrylate/2-hydroxypropyl methacrylate copolymers), anionic polymers (e.g., polyacrylic acids, crosslinked polyacrylic acids, vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and acrylic acid/ethyl acrylate/N-tert-butyl acrylamide terpolymers), thickening agents (e.g., agar-agar, guar gum, alginates, xanthan gum, gum arabic, karaya gum, carob seed meal, linseed gums, dextrans), cellulose derivatives (e.g., methyl cellulose, hydroxyalkyl cellulose and carboxymethyl cellulose), starch fractions and derivatives (e.g., amylose, amylopectin and dextrins), clays such as bentonite or fully synthetic hydrocolloids such as polyvinyl alcohol, texturizing agents such as maleic acid and lactic acid, hair-conditioning compounds such as phospholipids (e.g., soya lecithin, egg lecithin and cephalins), protein hydrolysates, particularly elastin, collagen, keratin, milk protein, soya protein and wheat protein hydrolysates, condensation products thereof with fatty acids and quaternized protein hydrolysates, perfume oils, active ingredients to improve the fiber structure, defoaming agents such as silicones, dyes to color the agent, anti-dandruff active ingredients (e.g., piroctone olamine, zinc omadine and climbazole), light stabilizers, particularly derivatized benzophenones, cinnamic acid derivatives and triazines, active ingredients such as allantoin, pyrrolidone carboxylic acids and salts thereof as well as bisabolol, vitamins, provitamins and vitamin precursors, particularly those of groups A, B₃, B₅, B₆, C, E, F and H, plant extracts, cholesterol, consistency modifiers such as sugar esters, polyol esters or polyol alkyl ethers, fats and waxes (e.g., spermaceti wax, beeswax, montan wax and paraffins), fatty acid alkanol amides, swelling and penetrating substances such as glycerol, propylene glycol monoethyl ethers, carbonates, hydrogen carbonates, guanidines, ureas as well as primary, secondary and tertiary phosphates, opacifiers (e.g., latex, styrene/PVP and styrene/acrylamide copolymers), pearlescent agents, pigments, stabilizing agents for hydrogen peroxide and other oxidizing agents, blowing agents (e.g., propane-butane mixtures, N₂O, dimethyl ether, CO₂ and air), and antioxidants.

Agents according to the invention can contain the ingredients in a suitable aqueous, alcoholic or aqueous-alcoholic carrier. For hair bleaching, such carriers include creams, emulsions, gels or surfactant-containing foaming solutions such as shampoos, foam aerosols or other preparations suitable for use on the hair. It is, however, also possible to provide a formulation in powder or tablet form, which is preferred for coloring and/or bleaching agents.

With respect to the present invention, aqueous-alcoholic solutions refer to aqueous solutions containing 3 to 70 wt. % of a C₁ to C₄ alcohol, particularly ethanol or isopropanol. Agents according to the invention can further contain additional organic solvents such as methoxybutanol, benzyl alcohol, ethyl diglycol or 1,2-propylene glycol. All water-soluble organic solvents are preferred here.

Preferred agents can additionally contain a non-aqueous solvent, with particularly preferred agents containing the solvent in a concentration of 0.1 to 30% by weight, preferably in a concentration of 1 to 20% by weight, most particularly preferably in a concentration of 2 to 10% by weight, based on total weight of the agent.

In further preferred agents, the solvent is chosen from ethanol, n-propanol, isopropanol, n-butanol, propylene glycol, n-butylene glycol, glycerol, diethylene glycol monoethyl ether, diethylene glycol mono-n-butyl ether, phenoxyethanol and benzyl alcohol, as well as mixtures thereof.

Agents according to the invention can also contain dyes and/or dye intermediates, and therefore be provided as agents having both bleaching and coloring action. Such agents are described below as “coloring agents”, “bleaching coloring agents” or “coloring and bleaching agents”.

Oxidative coloring of fibers can occur in the presence of oxidation dye intermediates with atmospheric oxygen. A chemical oxidizing agent is preferably used, however, particularly if a bleaching effect on human hair is sought in addition to the coloring effect. This bleaching effect may be sought regardless of the dyeing method. The presence of oxidation dye intermediates is therefore not an absolute prerequisite for use of oxidizing agents in the agents. Persulfates, chlorites, and particularly hydrogen peroxide or addition products thereof with urea, melamine and sodium borate are suitable as oxidizing agents.

According to the invention, however, the oxidization coloring agent can also be applied to hair together with a catalyst which activates oxidation of the dye intermediates, for example, through atmospheric oxygen. Such catalysts include metal ions, iodides, quinones or certain enzymes.

Suitable metal ions include Zn²⁺, Cu²⁺, Fe²⁺, Fe³⁺, Mn⁴⁺, Li⁺, Mg²⁺, Ca²⁺ and Al³⁺. Zn²⁺, Cu²⁺ and Mn²⁺ are particularly suitable. Metal ions can be used in the form of any physiological tolerable salt or in the form of a complex compound. Preferred salts are acetates, sulfates, halides, lactates and tartrates. Use of these metal salts can both accelerate the development of the color and selectively influence the color tint.

Suitable enzymes include peroxidases, which can significantly strengthen the action of small amounts of hydrogen peroxide. Furthermore, such enzymes are suitable which oxidize the oxidation dye intermediates directly by means of atmospheric oxygen (e.g., laccases), or which produce small amounts of hydrogen peroxide in situ and activate oxidation of the dye intermediates biocatalytically in this way. Particularly suitable catalysts for oxidation of the dye intermediates are the so-called 2-electron oxidoreductases in combination with their specific substrates, pyranose oxidase (with D-glucose or galactose for example), glucose oxidase (with D-glucose), glycerol oxidase (with glycerol), pyruvate oxidase (with benzotartaric acid or salts thereof), alcohol oxidase (with alcohol such as MeOH, EtOH), lactate oxidase (with lactic acid), tyrosinase oxidase (with tyrosine), uricase (with urea), choline oxidase (with choline) and amino acid oxidase (with amino acids).

If oxidizing agents are used, the bleaching and/or coloring agent is conveniently prepared directly before use by mixing the preparation containing the oxidizing agent with the preparation containing compounds of formulae (I) and (II) and optionally dye intermediates. The ready-to-use bleaching and/or hair coloring preparation formed by this process preferably has a pH in the range from 6 to 12. Use of the bleaching and/or hair coloring agent in a weakly alkaline environment is particularly preferred. Application temperatures can be in a range from 15 to 40° C. After a contact time of 5 to 45 minutes, the hair coloring agent is removed by rinsing the hair. There is no need to wash with a shampoo afterwards if a highly surfactant-containing carrier (e.g., a coloring shampoo) was used.

For hair that is difficult to dye, an agent according to the invention can be applied to the hair optionally with additional dye intermediates, but also without prior premixing with the oxidizing component. After a contact time of 20 to 30 minutes, the oxidizing component is then applied, optionally after an intermediate rinsing. After a further contact time of 10 to 20 minutes the hair is then rinsed and optionally shampooed. In this embodiment, the corresponding agent is adjusted according to a first variant wherein prior application of the dye intermediates is intended to bring about better penetration into the hair, to a pH of approximately 4 to 7. According to a second variant, oxidation by air is first sought, wherein the applied agent preferably has a pH of 7 to 10. In the subsequent accelerated post-oxidation, use of acidified peroxydisulfate solutions as oxidizing agents can be preferred.

Agents according to the invention can additionally contain further ingredients. Use of certain metal ions or complexes can be preferred, for example, to maintain intensive colors. Agents additionally containing Cu, Fe, Mn, Ru ions or complexes of these ions are preferred here.

Preferred agents according to the invention additionally contain Cu, Fe, Mn, Co, Ce, V, Ru ions or complexes of these ions, wherein particularly preferred agents contain 0.0001 to 2.5 wt. %, preferably 0.001 to 1 wt. %, of at least one compound from copper chloride (CuCl₂), copper sulfate (CuSO₄), iron(II) sulfate, manganese(II) sulfate, manganese(II) chloride, cobalt(II) chloride, cerium sulfate, cerium chloride, vanadium sulfate, manganese dioxide (MnO₂).

Preferred agents according to the invention can additionally contain one or more chelating agents chosen from—
(i) polycarboxylic acids, wherein the sum of carboxyl and optionally hydroxyl groups is at least 5 (in particular EDTA and salts thereof);
(ii) nitrogen-containing mono- or polycarboxylic acids;
(iii) geminal diphosphonic acids;
(iv) amino phosphonic acids;
(v) phosphonopolycarboxylic acids;
(vi) cyclodextrins;
   wherein preferred agents contain phosphonates, preferably hydroxyalkane or aminoalkane phosphonates, and particularly 1-hydroxyethane-1,1-diphosphonate (HEDP) or the disodium or tetrasodium salt thereof and/or ethylene diamine tetramethylene phosphonate (EDTMP) or the hexasodium salt thereof and/or diethylene triamine pentamethylene phosphonate (DTPMP) or the heptasodium or octasodium salt thereof.

As previously mentioned, agents according to the invention can be not only pure bleaching agents (i.e., as lightening agents), but also coloring and bleaching agents that color the keratin fibers at the same time as bleaching them. To this end, such agents contain at least one dye intermediate, preferably an oxidation dye intermediate, and/or at least one direct dye.

In addition to their function as bleaching agents, agents according to the invention can be coloring agents, that is, agents which change the color of keratin fibers. Of these, oxidation coloring agents are particularly preferred. Oxidation coloring agents according to the invention contain at least one coupler component and at least one developer component. Coupler and developer components are also known as oxidation dye intermediates. Oxidation coloring agents according to the invention can also contain direct dyes as tints.

Preferred agents for dyeing and/or bleaching keratin fibers thus can contain at least one oxidation dye intermediate of the developer type and/or coupler type.

If substrates are to be lightened or bleached, dyes coloring the substrate are mostly decolorized by oxidation using corresponding oxidizing agents, such as hydrogen peroxide.

In one embodiment for color change, agents of the present invention can be combined with at least one color-changing component. Color-changing components according to the present invention are preferably chosen from at least one oxidation dye intermediate of the developer component type and optionally additionally at least one coupler component and/or from at least one direct dye.

Preferred developer components include at least one compound from p-phenylene diamine, p-toluoylene diamine, 2-(β-hydroxyethyl)-p-phenylene diamine, 2-(α,β-dihydroxyethyl)-p-phenylene diamine, N,N-bis-(β-hydroxyethyl)-p-phenylene diamine, N-(4-amino-3-methylphenyl)-N-[3-(1H-imidazol-1-yl)propyl]amine, N,N-bis-(B-hydroxyethyl)-N,N′-bis-(4-aminophenyl)-1,3-diaminopropan-2-ol, bis-(2-hydroxy-5-aminophenyl)methane, 1,3-bis-(2,5-diaminophenoxy)propan-2-ol, N,N′-bis-(4-aminophenyl)-1,4-diazacycloheptane, 1,10-bis-(2,5-diaminophenyl)-1,4,7,10-tetraoxadecane, p-aminophenol, 4-amino-3-methylphenol, 4-amino-2-aminomethylphenol, 4-amino-2-(α,β-dihydroxyethyl)phenol and 4-amino-2-(diethylaminomethyl)phenol, 4,5-diamino-1-(β-hydroxyethyl)pyrazole, 2,4,5,6-tetraminopyrimidine, 4-hydroxy-2,5,6-triaminopyrimidine, 2-hydroxy-4,5,6-triaminopyrimidine, as well as the physiologically tolerable salts of these compounds.

With respect to oxidative dyeing, coupler components develop no significant color on their own, but always need the presence of developer components. It is therefore preferable that, with use of at least one developer component, at least one coupler component is also used.

Preferred coupler components include m-aminophenol, 5-amino-2-methylphenol, 3-amino-2-chloro-6-methylphenol, 2-hydroxy-4-aminophenoxyethanol, 5-amino-4-chloro-2-methylphenol, 5-(2′-hydroxyethyl)-amino-2-methylphenol, 2,4-dichloro-3-aminophenol, o-aminophenol, m-phenylene diamine, 2-(2,4-diaminophenoxy)ethanol, 1,3-bis-(2,4-diaminophenoxy)propane, 1-methoxy-2-amino-4-(2′-hydroxyethylamino)benzene, 1,3-bis(2,4-diaminophenyl)propane, 2,6-bis-(2′-hydroxyethylamino)-1-methylbenzene, 2-({3-[(2-hydroxyethyl)amino]-4-methoxy-5-methylphenyl}amino)ethanol, 2-({3-[(2-hydroxyethypamino]-2-methoxy-5-methylphenyl}amino)ethanol, 2-(({3-[(2-hydroxyethypamino]-4,5-dimethylphenyl}amino)ethanol, 2-[3-morpholin-4-ylphenyl)amino]ethanol, 3-amino-4-(2-methoxyethoxy)-5-methylphenylamine, 1-amino-3-bis-(T-hydroxyethyl)aminobenzene, resorcinol, 2-methyl resorcinol, 4-chlororesorcinol, 1,2,4-trihydroxybenzene, 2-amino-3-hydroxypyridine, 3-amino-2-methylamino-6-methoxypyridine, 2,6-dihydroxy-3,4-dimethylpyridine, 3,5-diamino-2,6-dimethoxypyridine, 1-phenyl-3-methylpyrazol-5-one, 1-naphthene, 1,5-dihydroxynaphthalene, 2,7-dihydroxynaphthalene, 1,7-dihydroxynaphthalene, 1,8-dihydroxynaphthalene, 4-hydroxyindole, 6-hydroxyindole, 7-hydroxyindole, 4-hydroxyindoline, 6-hydroxyindoline, 7-hydroxyindoline or mixtures of these compounds or the physiologically tolerable salts of the aforementioned compounds.

The developer and coupler components are preferably used in an amount from 0.005 to 20 wt. %, preferably 0.1 to 5 wt. %, based on total weight of the ready-to-use oxidation coloring agent.

Developer and coupler components are generally used in approximately molar amounts to one another. Even if molar use has proven convenient, a certain excess of individual oxidation dye intermediates is not disadvantageous, such that developer components and coupler components can be in a molar ratio of 1:0.5 to 1:3, in particular 1:1 to 1:2.

Agents according to the invention can further contain at least one direct dye. These are dyes which attach directly to the hair and require no oxidative process to develop the color. Direct dyes include nitrophenylene diamines, nitroaminophenols, azo dyes, anthraquinones or indophenols.

Direct dyes are preferably used in an amount from 0.001 to 20 wt. %, based on the entire application preparation. Total amount of direct dyes is preferably at most 20 wt. %.

Direct dyes can be divided into anionic, cationic and non-ionic direct dyes.

Preferred anionic direct dyes include compounds known under the international names or trade names Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1 and Acid Black 52.

Preferred cationic direct dyes include cationic triphenylmethane dyes such as Basic Blue 7, Basic Blue 26, Basic Violet 2 and Basic Violet 14, aromatic systems substituted with a quaternary nitrogen group, such as Basic Yellow 57, Basic Red 76, Basic Blue 99, Basic Brown 16 and Basic Brown 17, as well as direct dyes containing a heterocycle having at least one quaternary nitrogen atom, such as cited in EP-A2-998 908, for example, in claims 6 to 11.

Compounds known under the names Basic Yellow 87, Basic Orange 31 and Basic Red 51 are most particularly preferred cationic direct dyes.

Non-ionic nitro and quinone dyes and neutral azo dyes in particular are suitable as non-ionic direct dyes.

Preferred non-ionic direct dyes include compounds known under the international names or trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, as well as 1,4-diamino-2-nitrobenzene, 2-amino-4-nitrophenol, 1,4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]-benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid and 2-chloro-6-ethylamino-4-nitrophenol.

It is not necessary for the direct dyes to be uniform compounds. Instead, the individual dyes can contain small amounts of further components arising from the manufacturing processes for the individual dyes, provided that they do not adversely influence the dyeing result or need to be excluded for other (e.g., toxicological) reasons.

Naturally occurring dyes such as found in henna red, henna neutral, henna black, chamomile flowers, sandalwood, black tea, alder buckthorn bark, sage, logwood, madder root, catechu, lotus and alkanet root, can also be used as direct dyes.

The present invention secondly provides a method for bleaching keratin fibers, particularly human hair, wherein an agent according to the invention is applied to keratinous fibers, left on the fibers for 5 to 60 minutes, and then rinsed out or washed out with a shampoo. In particular, the temperature during the contact time of 5 to 60 minutes is from 10° C. to 40° C., particularly 20° C. to 38° C.

In the context of this method, it can be preferred that—
a pretreatment agent M1 is optionally applied to the fibers,
an agent M2 is applied to the fibers, a further agent M3 optionally being added to the agent M2 before application, and
agent M2 is rinsed from the fibers after a time of 5 to 60 minutes,
and after treatment a post-treatment agent M4 is optionally applied to the fibers and rinsed off again after a contact time of a few minutes,
   wherein at least one of the agents M1, M2 or M3 or the mixture of agents M2 and M3 is an agent according to the invention.

Agents according to the invention can therefore be formulated as one-component agents (coloring and/or bleaching agent M2) or as two-component agents (M2+M3) and used accordingly. Separation into multi-component systems is useful where incompatibilities between the ingredients are to be expected or feared. In such systems the agent used is prepared by the consumer just prior to use by mixing the components together.

A dyeing and/or bleaching method in which compounds of general structure (I), alkanol amines of general structure (II), and hydrogen peroxide are initially separate is preferred here. The present invention therefore also provides a method for dyeing and bleaching human hair wherein an aqueous composition containing hydrogen peroxide is mixed with a composition containing at least one compound of general structure (I) and alkanol amines of general structure (II) to form an agent according to the invention, and this is then applied to the hair.

In a further embodiment of the method for bleaching and optionally dyeing human hair, a composition on an aqueous basis containing hydrogen peroxide is mixed with a further agent containing preferably at least one alkalizing agent and/or direct hair dye and/or at least one oxidation dye intermediate and an agent containing the compounds of general structure (I) and formula (II) to form a homogeneous composition, and this is then applied to the hair.

The invention thirdly provides for use of agents of according to the invention for bleaching keratinous fibers, particularly human hair.

All that has been stated with respect to agents according to the invention applies with necessary alterations to further preferred embodiments of methods according to the invention and use according to the invention.

### EXAMPLES

### 1.0 Synthesis Example

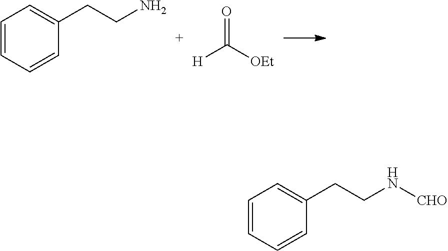

### 1.1 Synthesis of N-(2-phenylethyl)formamide

100.0 g (0.83 mol) of phenylethylamine and 187.0 g (2.07 mol) of ethyl formate were refluxed together for 12 hours. The excess ethyl formate was removed under vacuum in a rotary evaporator. A virtually colorless oil remained as residue, which was used in the next stage with no further purification; yield: 122.2 g (99.3%); ¹H-NMR (400 MHz, DMSO-d₆): δ [ppm]=2.72 (t, 2H); 3.45 (t, 2H); 7.19-7.31 (m, 5H); 8.00 (s, 1H); 8.10 (br, NH); ¹³C-NMR (400 MHz, DMSO-d₆): δ [ppm]=35.0; 38.7; 125.9; 128.0; 128.2; 139.1; 160.1.

### 1.2 Synthesis of 3,4-dihydroisoquinoline

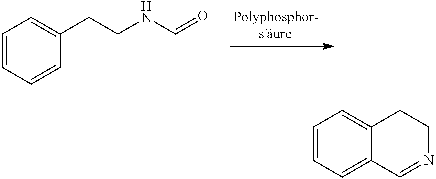

490.0 g (5.00 mol) of polyphosphoric acid were heated to 80° C. until it could be thoroughly mixed with a metal stirrer. Then 84.0 g (0.56 mol) of N-(2-phenylethyl)formamide from stage 1 were added at 80° C. while stirring and the mixture was heated to 160° C. for 12 hours. After the reaction the mixture was poured onto 1000 ml of iced water and then stirred for 2 hours at room temperature. A pH of 12.0 was established with a 5-molar, aqueous sodium hydroxide solution. The aqueous phase was extracted with methyl tert-butyl ether. The combined organic phases were dried with magnesium sulfate and completely evaporated in a rotary evaporator, resulting in a dark brown oil. The oil was distilled under vacuum (40 mbar/115° C.) and accumulated in the form of a clear, light-brown liquid, which was used in the third stage. Yield: 56.3 g (76.1%); ¹H-NMR (400 MHz, DMSO-d₆): δ [ppm]=2.68 (t, 2H); 3.65 (t, 2H); 7.23 (d, 1H); 7.34 (m, 2H); 7.41 (d, 1H); 8.34 (s, 1H); ¹³C-NMR (400 MHz, DMSO-d₆): δ [ppm]=25.4; 48.0; 127.5; 127.8; 128.9; 131.5; 137.0; 160.7.

### 1.2 Synthesis of N-methyl-3,4-dihydroisoquinoline-p-toluenesulfonate (A1)

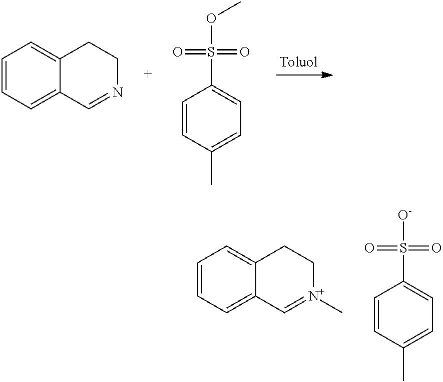

56.0 g (0.43 mol) of 3,4-dihydroisoquinoline from stage 2 were added to a solution of 80.0 g (0.43 mol) of p-toluene sulfonic acid methyl ester in 250 ml of toluene. The reaction mixture was stirred for three hours at 60° C., during which time the solution gradually became turbid. The solid which precipitated out after cooling was filtered off and dried under vacuum. Yield: 125.6 g (92.7%); ¹H-NMR (400 MHz, DMSO-d₆): δ [ppm]=2.25 (s, 3H); 3.18 (t, 2H); 3.72 (s, 3H); 4.01 (t, 3H); 7.09 (d, 2H); 7.20 (m, 2H); 7.52 (d, 2H); 7.58 (m, 1H); 7.79 (m, 1H); 9.23 (s, 1H); ¹³C-NMR (400 MHz, DMSO-d₆): δ [ppm]=24.2; 26.8; 50.0; 52.7; 126.0; 127.3; 130.6; 130.8; 132.3; 136.0; 139.1; 140.1; 140.7; 148.1; 169.1.

### 2.0—Bleaching Example—

2,1—Bleaching with Hydrogen Peroxide—

2.1.1—Preparation of a Bleaching Cream—

Bleaching creams were prepared from the list of ingredients as follows—

| | wt. % | | | | |
|---|---|---|---|---|---|
| Raw material | C1 | C2 | C3 | C4 | I |
| Hydrenol D | 6.9 | 6.9 | 6.9 | 6.9 | 6.9 |
| Lorol techn. | 2.5 | 2.5 | 2.5 | 2.5 | 2.5 |
| Eumulgin B1 | 0.6 | 0.6 | 0.6 | 0.6 | 0.6 |
| Eumulgin B2 | 0.6 | 0.6 | 0.6 | 0.6 | 0.6 |
| Akypo Soft 45 NV | 10.0 | 10.0 | 10.0 | 10.0 | 10.0 |
| Plantacare 1200 UP | 2.0 | 2.0 | 2.0 | 2.0 | 2.0 |
| Texapon K 14 S 70 C | 2.8 | 2.8 | 2.8 | 2.8 | 2.8 |
| Ammonium sulfate | 1.0 | 1.0 | 1.0 | 1.0 | 1.0 |
| Ascorbic acid | 0.1 | 0.1 | 0.1 | 0.1 | 0.1 |
| Sodium silicate 40/42 | 0.5 | 0.5 | 0.5 | 0.5 | 0.5 |
| Turpinal SL | 0.2 | 0.2 | 0.2 | 0.2 | 0.2 |
| Potassium hydroxide | 0.8 | 0.8 | 0.8 | 0.8 | 0.8 |
| Ammonia 25% | 7.1 | 7.1 | 7.1 | 7.1 | 7.1 |
| Imidazole | — | — | 2.0 | — | — |
| Monoethanolamine | — | — | — | 2.0 | — |
| Triethanolamine | — | — | — | — | 2.0 |
| N-Methyl-3,4- | — | 2.0 | 2.0 | 2.0 | 2.0 |
| dihydroisoquinoline-p- | | | | | |
| toluenesulfonate (A1) | | | | | |
| Water | to 100 | to 100 | to 100 | to 100 | to 100 |

Hydrenol ® D C₁₆-C₁₈ fatty alcohol (INCI name: Cetearyl alcohol) (Cognis)
Lorol ® tech. C₁₂-C₁₈ fatty alcohol (INCI name: Coconut alcohol) (Cognis)
Eumulgin ® B 1 Cetyl stearyl alcohol with approx. 12 EO units (INCI name: Ceteareth-12) (Cognis)
Eumulgin ® B2 Cetyl stearyl alcohol with approx. 20 EO units (INCI name: Ceteareth-20) (Cognis)
Akypo Soft 45 NV ® Lauryl alcohol-4.5-EO acetic acid sodium salt (min. 21% active substance content; INCI name: Sodium Laureth-6 Carboxylate) (Chem-Y)
Plantacare ® 1200 UP C₁₂₋₁₆ fatty alcohol-1,4-glucoside (approx. 50-53% active substance content; INCI name: Lauryl Glucoside, Aqua (Water)) (Cognis)
Texapon ® K 14 S 70 C Lauryl myristyl ether sulfate sodium salt (approx. 68% to 73% active substance content); INCI name: Sodium Myreth Sulfate) (Cognis)
Sodium silicate 40/42 Sodium silicate
Turpinal ® SL 1-Hydroxyethane-1,1-diphosphonic acid (approx. 58-61% active substance content; INCI name: Etidronic Acid, Aqua (Water)) (Solutia)

Hydrenol and Lorol were predispersed at elevated temperature. The other components were then incorporated one at a time while stirring, and the mixture then topped off with water to 100%.

Formulations C1, C2, C3 and C4 are comparative formulations not according to the invention. Formulation C1 is a standard formulation without bleach activator, formulation C2 is a standard formulation with bleach activator. Formulations C3 and C4 are formulations with bleach activator in combination with imidazole or monoethanolamine (2-aminoethanol). Formulation E represents a preparation according to the invention by way of example.

2.1.2—Mixing with the Developer Dispersion—

Each bleaching cream was mixed in a ratio of 1/:1 with a developer dispersion having the following composition. The pH of the final application mixture was from 9 to 10.2.

| Raw material | wt. % |
|---|---|
| Ammonia 25% | 0.62 |
| Dipicolinic acid | 0.10 |
| Disodium pyrophosphate | 0.03 |
| Turpinal SL | 1.50 |
| Texapon NSO | 2.00 |
| Dow Corning DB 110 A (non-ionic silicone emulsion) | 0.07 |
| Aculyn 33A (acrylic polymer) | 12.00 |
| Hydrogen peroxide 50% | 22.40 |
| Water | to 100 |

Turpinal ® SL 1-Hydroxyethane-1,1-diphosphonic acid (approx. 58-61% active substance content; INCI name: Etidronic Acid, Aqua (Water)) (Solutia)
Texapon ® NSO Lauryl ether sulfate, sodium salt (approx. 27.5% active substance; INCI name: Sodium Laureth Sulfate) (Cognis)
Aculyn ® 33 Acrylic polymer (approx. 28% solids in water; INCI name: Acrylates Copolymer)

For the bleaching process, four times the amount of the final application mixture was applied to strands of dark blonde, light brown and dark brown hair (codes: Kerling 7/0, Fischbach & Miller 6923 and Kerling 2/0) weighing approx. 0.7 g. After the strands had been bleached for 30 minutes at 32° C. they were washed with a commercial shampoo and dried with a hairdryer.

2.1.3—Assessment of the Bleaching Power—

Each hair strand was measured by colorimetry before and after the bleaching process. The dL value calculated using the following formula was used as a measure of the bleaching power of each formulation—

dL=L_{after}−L_{before}

L_{after}=lightness of the strands after bleaching; L_{before}=lightness of the strands before bleaching.

Two measurements were performed for each formulation and each hair type, and the average was calculated from the individual values in each case. The higher the dL value, the better the bleaching power of the individual formulation.

Bleaching power on dark blonde strands (Kerling 7/0)

| dL (formu- | dL (formu- | dL (formu- | dL (formu- | dL (formu- |
|---|---|---|---|---|
| lation C1) | lation C2) | lation C3) | lation C4) | lation I) |
| 8.56 | 12.78 | 13.57 | 13.29 | 14.22 |

Bleaching power on light brown strands (Fischbach & Miller 6923)

| dL (formu- | dL (formu- | dL (formu- | dL (formu- | dL (formu- |
|---|---|---|---|---|
| lation C1) | lation C2) | lation C3) | lation C4) | lation I) |
| 11.35 | 12.82 | 15.84 | 12.82 | 16.41 |

Bleaching power on dark brown strands (Kerling 2/0)

| dL (formu- | dL (formu- | dL (formu- | dL (formu- | dL (formu- |
|---|---|---|---|---|
| lation C1) | lation C2) | lation C3) | lation C4) | lation I) |
| 5.16 | 6.52 | 8.39 | 7.54 | 9.46 |

### 3.0—Significance of the Results—

Bleaching action of the various formulations can be estimated by comparing the dL values. Compared to standard bleaching without a bleach activator (C1), bleaching power can be improved by addition of a bleach activator (C2). It is absolutely clear from the dL values that the bleaching result can be improved still further by addition of an active ingredient (C3, C4 and I). Of all the active ingredients used, alkanol amines according to the invention (exemplified by the triethanolamine used (formulation I)) consistently showed the best result with the highest dL values across all hair types tested.

We claim:

1. Agent for bleaching keratin fibers comprising in a cosmetic carrier:
at least one cationic 3,4-dihydroisoquinoline compound of the following general structure (I) 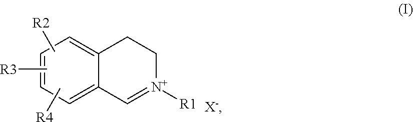
wherein
residue R1 is a C₁-C₆ alkyl group, C₂-C₆ alkenyl group, C₂-C₆ hydroxyalkyl group, C₁-C₆ alkoxy C₂-C₆ alkyl group, carboxy C₁-C₆ alkyl group, aryl C₁-C₆ alkyl group, C₁-C₆ dialkylamino C₂-C₆ alkyl group, heteroaryl C₁-C₆ alkyl group, 3-oxobutyl group, 2-oxopropyl group, aryl group or heteroaryl group,
residues R2, R3 and R4 are each independently hydrogen, hydroxyl group, amino group, di(C₁-C₆)alkylamino group, C₁-C₆ alkoxy group, halogen, nitro group, carboxy group, nitrile group, optionally substituted aryl group, C₂-C₆ alkenyl group, optionally substituted heteroaryl group or R2 and R3 together can form a further fused carbocyclic or heterocyclic ring, which can be saturated or unsaturated and can optionally be substituted by up to three substituents,
anion X— is a physiologically tolerable anion;
at least one alkanol amine of the following general structure (II), 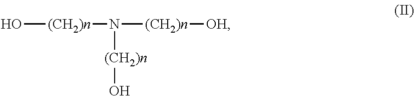
wherein n is a natural number from 2 to 6; and
hydrogen peroxide.

2. Agent according to claim 1, wherein residue R1 of general structure (I) is a C₁-C₆ alkyl group, a C₂-C₆ alkenyl group or a C₂-C₆ hydroxyalkyl group.

3. Agent according to claim 1, wherein residues R2, R3 and R4 of general structure (I) are each hydrogen.

4. Agent according to claim 1, wherein the physiologically tolerable anion X— denotes a halide ion, hydrogen sulfate, ½ sulfate, p-toluenesulfonate, benzenesulfonate or acetate.

5. Agent according to claim 1, wherein the at least one cationic compound of general structure (1) is chosen from the compounds
N-methyl-3,4-dihydroisoquinoline p-toluenesulfonate,
N-methyl-3,4-dihydroisoquinoline benzenesulfonate,
N-methyl-3,4-dihydroisoquinoline hydrogen sulfate,
N-allyl-3,4-dihydroisoquinoline p-toluenesulfonate,
N-allyl-3,4-dihydroisoquinoline benzenesulfonate,
N-allyl-3,4-dihydroisoquinoline bromide,
N-allyl-3,4-dihydroisoquinoline acetate,
3,4-dihydro-2-(3-hydroxypropyl)isoquinoline p-toluenesulfonate,
3,4-dihydro-2-(3-hydroxypropyl)isoquinoline benzenesulfonate,
3,4-dihydro-2-(3-hydroxypropyl)isoquinoline bromide,
3,4-dihydro-2-(3-hydroxypropyl)isoquinoline acetate,
3,4-dihydro-2-(2-hydroxyethyl)isoquinoline p-toluenesulfonate,
3,4-dihydro-2-(2-hydroxyethyl)isoquinoline benzenesulfonate,
3,4-dihydro-2-(2-hydroxyethyl)isoquinoline bromide, or
3,4-dihydro-2-(2-hydroxyethyl)isoquinoline acetate.

6. Agent according to claim 1, wherein n in the alkanol amines of general formula (II) is a whole number from 2 to 4.

7. Agent according to claim 1, wherein the alkanol amine of general formula (II) is a triethanolamine.

8. Agent according to claim 1, wherein cationic 3,4-dihydroisoquinoline derivatives of general structure (I) are present in an amount of from 0.03 to 65.00 mmol, based on 100 g of the agent.

9. Agent according to claim 1, wherein alkanol amines of general structure (II) are present in an amount of from 0.03 to 65.00 mmol, based on 100 g of the agent.

10. Agent according to claim 1 wherein the hydrogen peroxide is present in an amount of 0.5 to 12 wt. % (calculated as 100% H₂O₂), based on total weight of the agent.

11. Agent according to claim 1, wherein the pH of the ready-to-use application mixture is from 7 to 11.

12. Agent according to claim 1 further comprising at least one inorganic persulfate salt.

13. Agent according to claim 12, wherein the inorganic persulfate salt is chosen from ammonium peroxydisulfate, potassium peroxydisulfate and sodium peroxydisulfate.

14. Method for bleaching keratin fibers comprising applying an agent according to claim 1 to keratinous fibers, leaving the agent on the fibers for 5 to 60 minutes and rinsing out the agent or washing out the agent with a shampoo.
